(19) United States
(12) Patent Application Publication
(10) Pub. No.: US 2005/0176601 A1
(43) Pub. Date: Aug. 11, 2005

(54) Detergent cosmetic compositions comprising at least one aminosilicone and at least one drawing polymer, and uses thereof

(76) Inventors: Henri Samain, Bievres (FR); Geraldine Fack, Levallois (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 11/030,489

(22) Filed: Jan. 7, 2005

Related U.S. Application Data
(60) Provisional application No. 60/537,911, filed on Jan. 22, 2004.
(60) Provisional application No. 60/538,247, filed on Jan. 23, 2004.

(30) Foreign Application Priority Data
Jan. 7, 2004 (FR) 04 50030
Jan. 7, 2004 (FR) 04 50037

(51) Int. Cl. A61K 7/075

(52) U.S. Cl. 510/119

(57) ABSTRACT

The disclosure relates to novel washing compositions, such as, for example, hair compositions, comprising, in a cosmetically acceptable medium, at least one aminosilicone, at least one detergent surfactant chosen from anionic, nonionic and amphoteric surfactants and at least one drawing polymer. The present disclosure further relates to a process for treating hair using the compositions disclosed herein.

This application claims benefit of U.S. Provisional Application No. 60/537,911, filed Jan. 22, 2004, and U.S. Provisional Application No. 60/538,247, filed Jan. 22, 2004 and which are hereby incorporated by reference.

The present disclosure relates to cosmetic compositions with improved properties, intended for both cleaning, conditioning and styling keratin materials, such as, for example, the hair, comprising, in a cosmetically acceptable support, a washing base comprising at least one surfactant with detergent power, wherein at least one aminosilicone is also present in combination with high molecular weight polymers. The present disclosure also relates to a process for cleaning and/or conditioning and/or styling keratin materials comprising the application to the keratin materials of the compositions as disclosed herein.

It is common practice to use detergent hair compositions (or shampoos) based on standard surfactants of anionic, nonionic and/or amphoteric type such as, for example, anionic type, to clean and/or wash the hair. These compositions are applied to wet hair and the lather generated by massaging or rubbing with the hands can remove, after rinsing with water, the various types of soiling initially present on the hair.

These base compositions can have good washing power, but the intrinsic cosmetic properties associated therewith can nevertheless remain fairly poor, owing to the fact that the relatively aggressive nature of such a cleaning treatment can, in the long run, lead to more or less pronounced damage to the hair fiber. This damage can be associated, for instance, with the gradual removal of the lipids or proteins contained in or on the surface of this fiber.

Thus, in order to improve the cosmetic properties of the above detergent compositions, such as those that are intended to be applied to sensitized hair (i.e., hair that has been damaged or made brittle, for instance, due to the chemical action of atmospheric agents and/or hair treatments such as permanent-waving, dyeing or bleaching), it is now known to introduce additional cosmetic agents known as conditioners into these compositions, these conditioners being intended mainly to repair or limit the harmful or undesirable effects induced by the various treatments or aggressions to which the hair fibers can subjected more or less repeatedly. These conditioners may, of course, also improve the cosmetic behavior of natural hair.

The conditioners most commonly used to date in shampoos are cationic polymers, which can give washed, dry or wet hair at least one cosmetic property chosen from an ease of disentangling, softness and smoothness that can be markedly better than those that may be obtained with corresponding cleaning compositions from which cationic polymers are absent.

Moreover, it has for some time been sought to obtain conditioning shampoos that are capable of giving washed hair not only the cosmetic properties mentioned above but also, to a greater or lesser extent, at least one of styling, volume, shaping and hold properties. Such washing shampoos with improved general cosmetic properties are often referred to for simplicity as “styling shampoos”, and this term will be adopted in the description hereinbelow.

However, despite the progress made recently in the field of styling shampoos, these shampoos are not always satisfactory, and as such there is currently still a strong need to provide novel products that give better performance with respect to at least one of the cosmetic properties mentioned above. For example, it is usually necessary to use a styling product after conditioning, to give the hair a shape and to fix the style.

The present disclosure is directed towards satisfying such a need.

Thus it has now been found, surprisingly and unexpectedly, that by combining at least one aminosilicone, as defined below, and at least one anionic, nonionic, zwitterionic or amphoteric detergent surfactant, with at least one polymer that has a specific drawing power in detergent compositions, it is possible to substantially and significantly improve the styling and hold properties, while at the same time maintaining good intrinsic washing power and cosmetic properties.

For example, these compositions can make it possible to obtain very good hold and/or a certain amount of volume for the head of hair, i.e., a styling effect similar to that obtained with a fixing styling gel used after shampooing. It moreover may be found that the keratin fibers are strengthened.

All these discoveries form the basis of the present disclosure.

Accordingly, the present disclosure relates to novel compositions for washing keratin materials, such as the hair, comprising, in a cosmetically acceptable aqueous medium, at least one aminosilicone, at least one detergent surfactant chosen from anionic, nonionic and amphoteric surfactants and at least one drawing polymer with a drawing power of greater than 5 cm.

One aspect of the present disclosure is also the cosmetic use of the above compositions for cleansing, conditioning, caring for and styling keratin materials, for example, the hair and the eyelashes.

Another aspect of the present disclosure comprises a cosmetic process for treating keratin materials, such as keratin fibres, using the compositions disclosed herein.

Still another aspect of the present disclosure is the use of the composition as disclosed herein as a shampoo.

However, other characteristics and aspects of the present disclosure will emerge even more clearly upon reading the description that follows, and also the concrete, but in no way limiting, examples intended to illustrate it.

As used herein, the term “drawing power of a polymer” is understood to mean the length of polymer yarn obtained at the breaking point of the yarn according to the procedure defined below.

The drawing power of the polymers that may be used as disclosed herein is the power measured for a composition comprising (% by weight):

| | | |
|---|---|---|
| Sodium lauryl ether sulfate oxyethylenated | | 12.5% |
| with 2.2 mol of ethylene oxide | | |
| Cocoamidopropylbetaine | | 2.5% |
| Test polymer | | 1% |
| Water | qs | 100% |

The drawing power is measured using a TA-TX2 texture analyser (Rheo/stable Micro Systems).

The measurement is performed after compression of the product:
Displacement of the disc (35 mm aluminium cylinder) at a speed of 2.5 mm/s and detection of the compression strength
penetration into the product at the same speed to a depth of 10 mm
removal of the probe at a speed of 2.5 mm/s
measurement of the displacement of the probe and detection of the breaking point of the product yarn.

According to the present disclosure, the polymers with a drawing power of greater than 5 cm will also be referred to as “drawing polymers”.

The drawing polymers with a drawing power of greater than 5 cm may be chosen from, for example, either (a1) a dispersion of particles of at least one water-soluble polymer with a weight-average molecular mass of greater than 10⁶ in a saline aqueous solution, obtained by heterogeneous free-radical polymerization of water-soluble monomers with precipitation of the polymer formed, at least one of the monomers being cationic, or (a2) an aqueous saline solution of at least one water-soluble polymer with a weight-average molecular mass of greater than 10⁶, obtained by heterogeneous free-radical polymerization of water-soluble monomers with precipitation of the polymer formed, at least one of the monomers being cationic.

The water-soluble polymers may, in one embodiment, be cationic. As used herein, the term “cationic polymer” includes polymers comprising cationic monomers and possibly nonionic monomers.

As disclosed above, the synthesis of the high molecular weight water-soluble drawing polymers, used in the present disclosure, takes place by heterogeneous free-radical polymerization of water-soluble monomers comprising at least one ethylenic unsaturation. The polymerization takes place in an aqueous solution of a mineral electrolyte (salt) having an ionic strength that is sufficient to cause precipitation of the polymer formed as soon as the polymer has reached a certain molecular mass. This polymerization technique thus allows, by virtue of the well-known phenomenon of salting out, the preparation of saline aqueous dispersions of water-soluble polymer particles. The polymers thus synthesized are distinguished by a high weight-average molecular mass, which is greater than 10⁶.

The technique of heterogeneous free-radical polymerization in an aqueous medium with precipitation of the polymer formed is described, for example, in U.S. Pat. No. 4,929,655, in European Patent Application EP 0 943 628 or in International Patent Application WO 02/34796.

To ensure the stability of the dispersions of polymer particles during the synthesis and during storage, the polymerization may be performed in the presence of a dispersant. This dispersant can be, for example, be a polyelectrolyte, which, unlike the high molecular weight polymer used in the present disclosure, is soluble in the aqueous polymerization medium of high ionic strength.

This dispersing polyelectrolyte can have a charge identical to that of the polymer synthesized, in other words, for the synthesis of cationic polyelectrolytes, a cationic dispersing polyelectrolyte can be used.

Among the dispersants that may be used as disclosed herein, non-limiting mention may be made of the cationic polyelectrolytes obtained by polymerization of 50 to 100 mol % of at least one cationic monomer chosen from the salts, for example, the hydrochlorides or sulfates, dimethylaminoethyl (meth)acrylate, N-dimethylaminopropyl(meth)-acrylamide, di(meth)allylamine, (meth)acryloyloxy-ethyltrimethylammonium chloride, (meth)acrylamido-propyltrimethylammonium chloride and dimethyldiallyl-ammonium chloride, and of 50 to 0 mol % of acrylamide. A polyamine such as a polyalkyleneamine may also be used.

The dispersant may, for example, be used in an amount ranging from 1% to 10% by weight relative to the total weight of the monomers to be polymerized.

The saline aqueous solution that serves as synthesis and dispersion medium for the high molecular weight water-soluble drawing polymer is a solution of at least one mineral salt which may be chosen from divalent anionic salts. Such anionic salts may be chosen from ammonium sulfate, ammonium hydrogen sulfate, sodium sulfate, sodium hydrogen sulfate, magnesium sulfate, ammonium sulfate, magnesium hydrogen sulfate, aluminium sulfate and aluminium hydrogen sulfate. In one embodiment of the present disclosure, the anionic salts are chosen from ammonium sulphate and sodium sulphate.

The concentration of the at least one salt can be sufficient to induce the precipitation of the water-soluble polymer formed in the polymerization medium, and may be up to the saturation concentration of each salt. To obtain such a precipitation, the salt concentration may range from 10% to 50%, for example, greater than 15% and less than 50%, by weight relative to the total weight of the polymer solution or dispersion. The saline aqueous solution may also comprise monovalent salts such as sodium chloride and ammonium chloride.

The heterogeneous free-radical polymerization in aqueous medium as described above can be accompanied by a large increase in the viscosity of the reaction medium, which may be reflected by difficulties in stirring, a lack of homogeneity of the reaction medium and an increase in the particle size of the polymer particles formed. To prevent such an increase in viscosity, it has been proposed, in European Patent Application EP 0 943 628, to add to the polymerization medium at least one agent for preventing the increase in viscosity of the reaction medium during polymerization.

The high molecular weight water-soluble drawing polymers as disclosed herein may, for example, be prepared in the presence of at least one agent for preventing the increase in viscosity.

The at least one agent for preventing the increase in viscosity of the reaction medium are chosen, for example, from:
(1) polycarboxylic acids and salts thereof,
(2) polyphenols,
(3) cyclic compounds comprising a hydroxyl group and a carboxyl group, or salts thereof,
(4) gluconic acid or salts thereof,
(5) the reaction products obtained by reacting a methoxyhydroquinone and/or a cationic (meth)acrylic monomer with a free-radical-generating compound, under an oxidizing atmosphere,
(6) the reaction products obtained by reacting a cationic (meth)acrylic polymer with a free-radical-generating compound, under an oxidizing atmosphere,
(7) the reaction products obtained by reacting a cationic (meth)acrylic polymer with an oxidizing agent,
   and mixtures thereof.

The addition of at least one agent for preventing the increase in viscosity as described above makes it possible to perform the polymerization of the water-soluble monomers described above with a low-power stirrer while at the same time avoiding the formation of coarse particles. The agents for preventing an increase in viscosity can be, for example, soluble in the aqueous reaction medium.

Non-limiting examples of (1) polycarboxylic acids and salts thereof that may be mentioned include oxalic acid, adipic acid, tartaric acid, malic acid and phthalic acid, and the salts thereof.

Non-limiting examples of polyphenols (2) that may be mentioned, for instance, include resorcinol and pyrogallol.

Non-limiting examples of (3) cyclic compounds comprising a hydroxyl group and a carboxyl group, or salts thereof that may be mentioned include m-hydroxybenzoic acid, p-hydroxybenzoic acid, salicylic acid, gallic acid and tannic acid, and the salts of these acids.

Non-limiting examples of (4) gluconic acid or salts thereof that may be mentioned include sodium gluconate, potassium gluconate, ammonium gluconate and various amine salts of gluconic acid.

Non-limiting examples of the reaction products (5) obtained by reacting a methoxyhydroquinone and/or a cationic (meth)acrylic monomer with a free-radical-generating compound, under an oxidizing atmosphere that may be mentioned include those obtained by reacting a free-radical-generating compound, under a stream of oxygenated gas, in a solution containing methoxyhydroquinone and/or a cationic (meth)acrylic monomer. The free-radical-generating compound may be an initiator commonly used for free-radical polymerization. Non-limiting examples that may be mentioned include water-soluble azo initiators such as 2,2′-azobis(2-amidinopropane) hydrochloride sold, for example, under the name V-50 by the company Wako Chemical Industries, or 2,2′-azobis[2-(2-imidazolin-2-yl)propane] hydrochloride sold, for example, under the trade name VA-044 by the company Wako Chemical Industries, or an initiator from the group of water-soluble redox agents, such as the ammonium persulphate/sodium hydrogen sulphite combination.

The agents for preventing an increase in viscosity such as (6) the reaction products obtained by reacting a cationic (meth)acrylic polymer with a free-radical-generating compound, under an oxidizing atmosphere, may be obtained by reacting a free-radical initiator, under a oxygenated atmosphere, with a dispersant as disclosed herein. The polymerization initiator may be a water-soluble azo initiator or a water-soluble redox agent as described above.

The reaction products (7) obtained by reacting a cationic (meth)acrylic polymer with an oxidizing agent may be obtained in the form of oxidized polymers of low molecular mass by oxidation of a cationic dispersant according to the present disclosure obtained by polymerization of a cationic (meth)acrylic monomer, using hydrogen peroxide or a halogen as oxidizing agent.

Among the cationic (meth)acrylic monomers used for the preparation of agents for preventing an increase in viscosity of types (5), (6), and (7), non-limiting examples that may be mentioned include dimethylaminoethyl (meth)acrylate hydrochloride or sulfate, (meth)acryloyloxyethyl-trimethylammonium chloride, (meth)acryloyloxyethyl-dimethylbenzylammonium chloride, the hydrochloride or sulfate derived from N-dimethylaminopropyl(meth)-acrylamide, (meth)acrylamidopropyltrimethylammonium chloride, dimethylaminohydroxypropyl (meth)acrylate chloride or sulfate, (meth)acryloyloxyhydroxypropyl-trimethylammonium chloride and (meth)acryloxyloxyhydroxypropyldimethylbenzylammonium chloride.

The agents for preventing an increase in viscosity as set forth above may be used alone or as a mixture, in an amount ranging, for example, from 10 ppm to 10 000 ppm relative to the total weight of the reaction solution.

The water-soluble monomers polymerized by heterogeneous free-radical polymerization to obtain the high molecular weight water-soluble drawing polymers are monomers comprising at least one ethylenic double bond, for example a vinyl, acrylic or allylic double bond. They may be cationic, anionic or nonionic and may be used as a mixture, at least one being cationic.

Non-limiting examples of water-soluble anionic monomers that may be mentioned include acrylic acid, methacrylic acid, acrylamido-2-methylpropanesulfonic acid and itaconic acid. The anionic monomers are at least partially neutralized in the form of a salt of an alkali metal (for example sodium or potassium), of an alkaline-earth metal, of ammonium or of an organic amine such as an alkanolamine, such as, for example, ethanolamine.

Non-limiting examples of water-soluble nonionic monomers that may be mentioned include acrylamide, methacrylamide, N-vinylformamide, N-vinylacetonamide, hydroxypropyl acrylate and hydroxypropyl methacrylate.

The at least one water-soluble cationic monomers can be chosen, for example, from di(C₁₋₄ alkyl)diallylammonium salts and the compounds of formula (I) 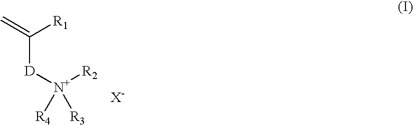
wherein:
R₁ is chosen from a hydrogen atom and methyl groups;
R₂ and R₃, which may be identical or different, each are chosen from a hydrogen atom and a linear and branched C₁₋₄ alkyl groups;
R₄ is chosen from a hydrogen atom, a linear and branched C₁₋₄ alkyl groups and aryl groups;
D is chosen from units of the following formula: 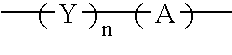
   wherein Y is chosen from amide (—CO—NH—), ester (—O—CO— and —CO—O—), urethane (—O—CO—NH—) and urea (—NH—CO—NH—) functional groups;
A is chosen from a linear, branched and cyclic C₁₋₁₀ alkylene group, which may be substituted or interrupted with a divalent aromatic or heteroaromatic ring, or which may be interrupted with a hetero atom chosen from O, N, S and P, and which may comprise a ketone, amide, ester, urethane or urea functional group;
n is equal to 0 or 1; and
X⁻ is chosen from an anionic counterion such as a chloride or sulfate ion.

Water-soluble cationic monomers useful herein may, for example, be chosen from dimethylaminoethyl (meth)acrylate hydrochloride or sulfate, (meth)acryloyloxyethyltrimethylammonium chloride, (meth)acryloyloxyethyldimethylbenzylammonium chloride, N-dimethylaminopropyl(meth)acrylamide hydrochloride or sulfate, (meth)acrylamidopropyltrimethylammonium chloride, (meth)acrylamidopropyldimethylbenzylammonium chloride, dimethylaminohydroxypropyl (meth)acrylate hydrochloride or sulfate, (meth)acryloyloxyhydroxypropyltrimethylammonium chloride, (meth)acryloyloxyhydroxypropyldimethylbenzylammonium chloride and dimethyldiallylammonium chloride.

In one embodiment of the present disclosure, the high molecular weight water-soluble polymer is obtained by heterogeneous free-radical polymerization of a monomer mixture comprising from 0 to 30 mol % of acrylic acid, from 0 to 95.5 mol % of acrylamide and from 0.5 to 100 mol % of at least one cationic monomer of formula (I).

For example, the drawing polymer may be obtained by heterogeneous free-radical polymerization of a monomer mixture comprising from 0 to 95.5 mol % of acrylamide and from 4.5 to 100 mol % of at least one cationic monomer of formula (I).

According to one embodiment of the present disclosure, the water-soluble polymers are obtained by polymerization of a monomer mixture comprising acrylic acid and a cationic monomer of formula (I), wherein the number of moles of cationic monomer of formula (I) is greater than the number of moles of acrylic acid.

Water-soluble polyelectrolytes may be, by non-limiting example, those polymerized using monomer mixtures comprising:
1) 10 mol % of acryloyloxyethyldimethylbenzyl-ammonium chloride and 90 mol % of acrylamide;
2) 30 mol % of acryloyloxytrimethylammonium chloride, 50 mol % of acryloyloxyethyldimethylbenzyl-ammonium chloride and 20 mol % of acrylamide;
3) 10 mol % of acryloyloxyethyltrimethylammonium chloride and 90 mol % of acrylamide;
4) 30 mol % of diallyidimethylammonium chloride and 70 mol % of acrylamide.

The drawing polymers, such as, for example, the water-soluble polymers, used in the present disclosure, have a weight-average molecular mass of greater than 1 000 000, for example, ranging from 1 000 000 to 50 000 000. This weight-average molecular mass is determined via the RSV (Reduced Specific Viscosity) method as defined in “Principles of Polymer Chemistry”, Cornell University Press, Ithaca, N.Y., 1953, Chapter VII: “Determination of Molecular Weight,” pages 266-316.

The concentration of the high molecular weight water-soluble polymer dispersion or solution can be, for example, chosen such that the concentration of the water-soluble polymer ranges from 0.01% to 10% by weight, for example, from 0.05% to 5% by weight relative to the total weight of the final composition.

The concentration of particles of the at least one water-soluble polymer as a dispersion in a saline aqueous solution in (a1) may range from 0.01% to 20% by weight, relative to the total weight of the dispersion.

The concentration of the at least one water-soluble polymer as a solution in (a2) may range from 0.01% to 20% by weight, relative to the total weight of the solution.

The at least one drawing polymer may, for example, be present in an amount ranging from 0.01% to 10% by weight, for example, from 0.05% to 5% by weight relative to the total weight of the final composition.

The at least one detergent surfactant is chosen from anionic, amphoteric, nonionic and zwitterionic surfactants.

Thus, according to the present disclosure, the at least one detergent surfactant may be present in an amount ranging from 4% to 50% by weight, for example, from 6% to 30% by weight and further, for example, from 8% to 25% by weight, relative to the total weight of the final composition.

The surfactants that are suitable for carrying out the present disclosure may be chosen from:

(i) Anionic Surfactant(s):

Non-limiting examples of anionic surfactants that may be used, alone or as mixtures, in the context of the present disclosure, may be chosen from salts (such as, for example alkaline salts, such as, for example, sodium salts, ammonium salts, amine salts, amino alcohol salts and magnesium salts) of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, the alkyl or acyl radical of all of these various compounds comprising, for example, from 12 to 20 carbon atoms. In one embodiment of the present disclosure, the aryl radical is chosen from phenyl and benzyl groups.

Among the anionic surfactants that may also be used, non-limiting mention may also be made of fatty acid salts such as the salts of oleic, ricinoleic, palmitic and stearic acids, coconut oil acid or hydrogenated coconut oil acid; acyl lactylates wherein the acyl radical ranges from 8 to 20 carbon atoms. Use may also be made of weakly anionic surfactants, such as alkyl-D-galactosideuronic acids and their salts, and also polyoxyalkylenated carboxylic ether acids and their salts, such as, for example, those ranging from 2 to 50 ethylene oxide groups, and mixtures thereof. Anionic surfactants of the polyoxyalkylenated carboxylic ether acid or salt type are, for example, those that chosen from formula (1)
R₁—(OC₂H₄)ₙ—OCH₂COOA (1)
wherein:
R₁ is chosen from alkyl, alkylamido and alkaryl groups;
n is an integer or decimal number (average value) ranging from 2 to 24, for example from 3 to 10, the alkyl radical comprising from 6 to 20 carbon atoms, and the aryl may, in some embodiments, be phenyl,
A is chosen from a hydrogen atom, ammonium, Na ions, K ions, Li ions, Mg ions, monoethanolamine residues and triethanolamine residues.

Mixtures of compounds of formula (1) may optionally also be used, for example, mixtures wherein the groups R₁ are different.

Compounds of formula (1) are sold, for example, by the company Chem Y under the name Akypo (NP40, NP70, OP40, OP80, RLM25, RLM38, RLMQ 38 NV, RLM 45, RLM 45 NV, RLM 100, RLM 100 NV, RO 20, RO 90, RCS 60, RS 60, RS 100, RO 50) or by the company Sandoz under the name Sandopan (DTC Acid, DTC).

(ii) Nonionic Surfactant(s):

Nonionic surfactants are likewise compounds that are well known per se (see “Handbook of Surfactants” by M. R. Porter, published by Blackie & Son (Glasgow and London), 1991, pp. 116-178). They may be chosen, for example, from polyethoxylated, polypropoxylated and polyglycerolated fatty alcohols, polyethoxylated, polypropoxylated and polyglycerolated fatty α-diols, polyethoxylated, polypropoxylated and polyglycerolated fatty alkylphenols and polyethoxylated, polypropoxylated and polyglycerolated fatty acids, all having a fatty chain comprising, for example, 8 to 18 carbon atoms, it being possible for the number of ethylene oxide or propylene oxide groups to range, for example, from 2 to 50 and for the number of glycerol groups to range, for example, from 2 to 30. Non-limiting mention may also be made of copolymers of ethylene oxide and of propylene oxide, condensates of ethylene oxide and of propylene oxide with fatty alcohols; polyethoxylated fatty amides having, for example, from 2 to 30 mol of ethylene oxide, polyglycerolated fatty amides comprising on average 1 to 5, such as, for example 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. In one embodiment of the present disclosure, the nonionic surfactants are chosen from alkylpolyglycosides.

(iii) Amphoteric or Zwitterionic Surfactant(s):

The amphoteric or zwitterionic surfactants may, by non-limiting example, be chosen from aliphatic secondary or tertiary amine derivatives wherein the aliphatic radical is chosen from linear and branched chains comprising from 8 to 18 carbon atoms and comprising at least one water-soluble anionic group (for example carboxylate, sulfonate, sulfate, phosphate or phosphonate); non-limiting mention may also be made of (C₈-C₂₀)-alkylbetaines, sulfobetaines, (C₈-C₂₀)alkylamido(C₁-C₆)alkylbetaines or (C₈-C₂₀)alkylamido(C₁-C₆)alkylsulfobetaines.

Among the amine derivatives, non-limiting mention may be made of the products sold under the name Miranol, as described in U.S. Pat. No. 2,528,378 and U.S. Pat. No. 2,781,354 and classified in the CTFA dictionary, 3rd edition, 1982, under the names Amphocarboxyglycinates and Amphocarboxypropionates, with the respective structures of formulae (2) and (3):
R₂—CONHCH₂CH₂—N⁺(R₃)(R₄)(CH₂COO⁻) (2)
wherein: R₂ is chosen from an alkyl radical of an acid R₂—COOH present in hydrolysed coconut oil, and heptyl, nonyl and undecyl radicals;
R₃ is a β-hydroxyethyl group; and
R₄ is a carboxymethyl group; and
   R₂′—CONHCH₂CH₂—N(B)(C) (3)
   wherein:
B is chosen from —CH₂CH₂OX′;
   wherein:
   X′ is chosen from —CH₂CH₂—COOH and a hydrogen atom;
C is chosen from —(CH₂)_{z}—Y′,
   wherein:
   z is equal to 1 or 2;
   Y′ is chosen from —COOH and —CH₂—CHOH—SO₃H;
R_{2′} is chosen from an alkyl radical of an acid R₂—COOH present in coconut oil or in hydrolysed linseed oil, an alkyl radical, such as, for example C₇, C₉, C₁₁, and C₁₃ alkyl radicals, C₁₇ alkyl radicals and their iso forms, and unsaturated C₁₇ radicals

For example, non-limiting mention may be made of the cocoamphocarboxyglycinate sold under the trade name Miranol C2M concentrate by the company Miranol.

Mixtures of surfactants such as, for example, mixtures of anionic surfactants, mixtures of anionic surfactants and of amphoteric, cationic or nonionic surfactants, or mixtures of cationic surfactants with nonionic or amphoteric surfactants, may be used in the compositions in accordance with the present disclosure, for example, a mixture comprising at least one anionic surfactant and at least one amphoteric surfactant.

The amount of the at least one anionic surfactant may, for example, range from 3% to 40% by weight relative to the total weight of the cosmetic composition. It may, by further example, range from 5% to 35% by weight, such as from 8% to 25% by weight.

The amount of at least one amphoteric and/or nonionic surfactant, when they are present, may, for example, range from 0.5% to 20% by weight such as, by further example, from 1% to 15% by weight, relative to the total weight of the composition.

As used herein, the term “aminosilicone” means any silicone comprising at least one primary, secondary or tertiary amine or a quaternary ammonium group. For example, aminosilicones may be chosen from:
(a) the polysiloxanes referred to in the CTFA dictionary as “amodimethicone” and of formula (VIII): 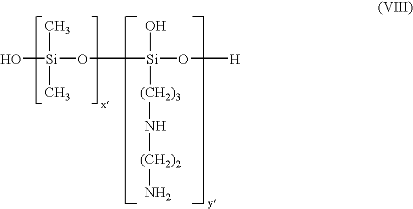
   wherein x′ and y′ are integers such that the number-average molecular weight ranges from 5000 to 500 000;
(b) aminosilicones of formula (IX):
   R′ₐG₃₋ₐ-Si(OSiG₂)ₙ-(OSiG_{b}R′_{2-b})ₘ—O—SiG₃₋ₐ—R′ₐ (IX)
   wherein:
G is chosen from a hydrogen atom, phenyl, OH and C₁-C₈ alkyl, for example methyl, groups;
a is an integer ranging from 0 to 3; where in some embodiments, a is 0;
b is equal to 0 or 1, where in some embodiments b is 1;
m and n are numbers such that the sum (n+m) may range, for example, from 1 to 2000, such as, by further example, from 50 to 150;
n, for example, ranges from 0 to 1999 such as, by further example, from 49 to 149; and
m, for example, ranges from 1 to 2000, such as, by further example, from 1 to 10;
R′ is a monovalent radical of formula —C_{q}H_{2q}L
wherein:
q is a number from 2 to 8; and

L is an optionally quaternized amine group chosen from:
—NR″—CH₂—CH₂-N′(R″)₂
—N(R″)₂
—N⊕(R″)₃A⁻
—NH⊕(R″)₂A⁻
—NH₂⊕(R″)A⁻
—N(R″)—CH₂—CH₂-N⊕R″H₂A⁻,
   wherein:
R″ may, for example, be chosen from hydrogen, phenyl, benzyl and saturated monovalent hydrocarbon-based radicals, for example an alkyl radical comprising from 1 to 20 carbon atoms; and
A⁻ is chosen from a halide ion such as, for example, fluoride, chloride, bromide or iodide.

A product corresponding to this definition is the silicone known as “trimethylsilyl amodimethicone”, of the formula (X): 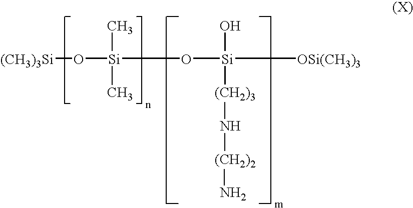
wherein n and m have the meanings given above (cf. formula IX).

Such polymers are described, for example, in European Patent Application EP-A-95238.

(c) aminosilicones of formula: 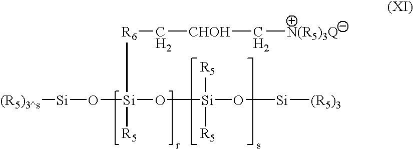
wherein:
R₅ is chosen from monovalent hydrocarbon-based radicals comprising from 1 to 18 carbon atoms, such as, for example, a C₁-C₁₈ alkyl or C₂-C₁₈ alkenyl radical, for example methyl;
R₆ is chosen from divalent hydrocarbon-based radicals, such as, for example, a C₁-C₁₈ alkylene radical or a divalent C₁-C₁₈, for example C₁-C₈, alkylenoxy radical linked to the Si via an SiC bond;
Q⁻ is chosen from anions such as a halide ion, such as, for example, chloride, and an organic acid salt (acetate, etc.);
r is a number ranging from 2 to 20 such as, for example, from 2 to 8;
s is a number ranging from 20 to 200 such as, for example, from 20 to 50.

Such aminosilicones are described in U.S. Pat. No. 4,185,087.

A silicone falling within this category is the silicone sold by the company Union Carbide under the name “Ucar Silicone ALE 56”.

d) quaternary ammonium silicones of formula (XII): 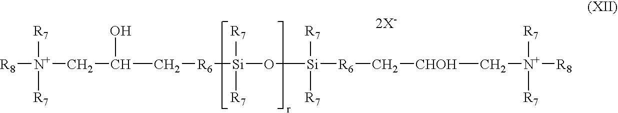
wherein:
R₇, which may be identical or different, are each chosen from a monovalent hydrocarbon-based radical comprising from 1 to 18 carbon atoms, such as, for example, a C₁-C₁₈ alkyl radical, a C₂-C₁₈ alkenyl radical and a ring comprising 5 or 6 carbon atoms, for example methyl;
R₆ is chosen from a divalent hydrocarbon-based radical, such as, for example, a C₁-C₁₋₈ alkylene radical and a divalent C₁-C₁₈, for example C₁-C₈, alkylenoxy radical linked to the Si via an SiC bond;
R₈, which may be identical and different, are each chosen from a hydrogen atom, a monovalent hydrocarbon-based radical comprising from 1 to 18 carbon atoms, such as, for example, a C₁-C₁₈ alkyl radical, a C₂-C₁₈ alkenyl radical and a radical —R₆—NHCOR₇;
X⁻ is an anion such as a halide ion, for example, chloride, and an organic acid salt (acetate, etc.); and
r is an average statistical value ranging from 2 to 200 such as, for example, from 5 to 100.

These silicones are described, for example, in Application EP-A-0 530974.

Silicones falling within this class are the silicones sold by the company Goldschmidt under the names Abil® Quat 3270, Abil® Quat 3272 and Abil® Quat 3474.

e) aminosilicones of formula (XIII): 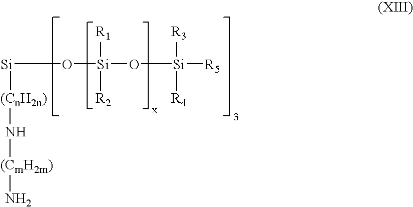
R₁, R₂, R₃ and R₄, which may be identical and different, are each chosen from C₁-C₄ alkyl radicals and phenyl groups,
R₅ is chosen from C₁-C₄ alkyl radicals and a hydroxyl group,
n is an integer ranging from 1 to 5,
m is an integer ranging from 1 to 5,
   and wherein x is chosen such that the amine number ranges from 0.01 to 1 meq/g.

According to the present disclosure, the aminosilicones may optionally be in the form of an oil, of aqueous, alcoholic and aqueous-alcoholic solutions, in the form of a dispersion and an emulsion.

The aminosilicones may be used in the form of emulsions, such as, for example, in the form of microemulsions and nanoemulsions.

The product sold under the name “Cationic Emulsion DC 929” by the company Dow Corning, which comprises, besides amodimethicone, a cationic surfactant derived from tallow fatty acids, referred to as Tallowtrimonium (CTFA), in combination with a nonionic surfactant, known under the name “Nonoxynol 10”, may be used for example.

The product sold under the name “Cationic Emulsion DC 939” by the company Dow Corning, which comprises, besides amodimethicone, a cationic surfactant, trimethylcetylammonium chloride, in combination with a nonionic surfactant, trideceth-12, may also be used for example.

Another commercial product which may be used according to the present disclosure is the product sold under the name “Dow Corning Q2 7224” by the company Dow Corning, comprising, in combination, the trimethylsilyl amodimethicone of formula (X), a nonionic surfactant of formula: C₈H₁₇—C₆H₄—(OCH₂CH₂)ₙ—OH wherein n=40, also known as octoxynol-40, another nonionic surfactant of formula: C₁₂H₂₅—(OCH₂—CH₂)ₙ—OH wherein n 6, also known as isolaureth-6, and glycol.

According to the present disclosure, all the silicones may also be used in the form of emulsions and microemulsions.

The silicones may be chosen from:
polysiloxanes comprising amine groups, such as amodimethicones and trimethylsilyl amodimethicones (CTFA 4th edition 1997).

According to the present disclosure, the aminosilicone(s) may be present in an amount ranging from 0.001% to 20% by weight, for example, from 0.01% to 10% by weight and further, for example, from 0.1% to 3% by weight relative to the total weight of the final composition.

According to one embodiment of the present disclosure, the compositions may also comprise at least one additional cationic or amphoteric polymer other than the drawing polymer according to the present disclosure.

The additional cationic polymers that may be used in accordance with the present disclosure may be chosen from all those already known per se as improving the cosmetic properties of the hair, i.e. such as, for example, those described in European Patent Application EP-A-0 337 354 and in French Patent Applications FR-A-2 270 846, 2 383 660, 2 598 611, 2 470 596 and 2 519 863, and having a suitable cationic charge density.

As used herein, the term “cationic polymer” is understood to mean any polymer comprising cationic groups and/and groups that may be ionized into cationic groups.

The additional cationic polymers may, for example, be chosen from those comprising units comprising primary, secondary, tertiary and/and quaternary amine groups that either may form part of the main polymer chain and may be borne by a side substituent directly attached thereto.

The additional cationic polymers used generally have a number-average and weight-average molar mass ranging from 500 to 5×10⁶, for example, from 103 to 3×10⁶.

The additional cationic polymers may be chosen from polyamine, polyamino amide and polyquaternary ammonium type.

The polymers of the polyamine, polyamino amide and polyquaternary ammonium type that may be used in accordance with the present disclosure include, for example, those described in French Patent Nos 2 505 348 and 2 542 997. Among these polymers, non-limiting mention may be made of:
(1) homopolymers and copolymers derived from acrylic and methacrylic esters and amides and comprising at least one of the units of the following formulae: 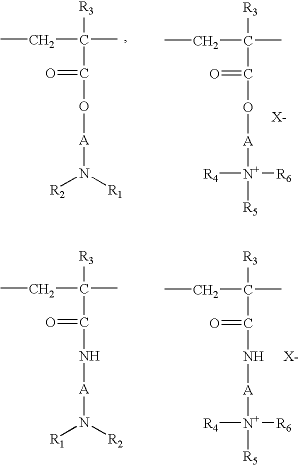
   wherein:
   R₃, which may be identical and different, is chosen from a hydrogen atom and CH₃ radicals;
   A, which may be identical and different, is chosen from linear and branched alkyl groups of 1 to 6 carbon atoms, for example, 2 and 3 carbon atoms, and hydroxyalkyl groups of 1 to 4 carbon atoms;
   R₄, R₅ and R₆, which may be identical and different, are each chosen from alkyl groups comprising from 1 to 18 carbon atoms and benzyl radicals, for example, an alkyl group comprising from 1 to 6 carbon atoms;
   R₁ and R₂, which may be identical and different, are each chosen from hydrogen atoms and alkyl groups comprising from 1 to 6 carbon atoms, for example, methyl and ethyl;
   X⁻ is an anion derived from a mineral and organic acid, such as a methosulfate anion and a halide such as chloride and bromide.

The copolymers of family (1) may also comprise at least one unit derived from comonomers that may be chosen from the family of acrylamides, methacrylamides, diacetone acrylamides, acrylamides and methacrylamides substituted on the nitrogen with lower (C₁-C₄) alkyls, acrylic and methacrylic acids and esters thereof, vinyllactams such as vinylpyrrolidone and vinylcaprolactam, and vinyl esters.

Thus, among these copolymers of family (1), non-limiting mention may be made of:
copolymers of acrylamide and of dimethylaminoethyl methacrylate quaternized with dimethyl sulfate and with a dimethyl halide, such as the product sold under the name Hercofloc by the company Hercules,
the copolymers of acrylamide and of methacryloyloxyethyltrimethylammonium chloride described, for example, in European Patent Application EP-A-080 976 and sold under the name Bina Quat P 100 by the company Ciba Geigy,
the copolymers of acrylamide and of methacryloyloxyethyltrimethylammonium methosulfate sold under the name Reten by the company Hercules,
quaternized and non-quaternized vinylpyrrolidone/dialkylaminoalkyl acrylate and methacrylate copolymers. These polymers are described in detail in French Patent Nos. 2 077 143 and 2 393 573,
dimethylaminoethyl methacrylate/vinylcaprolactam/vinyl pyrrolidone terpolymers,
vinylpyrrolidone/methacrylamidopropyldimethylamine copolymers,
and quaternized vinylpyrrolidone/dimethylaminopropylmethacrylamide copolymers.
(2) cationic polysaccharides, such as, for example, cationic celluloses and cationic galactomannan gums. The cationic polysaccharides also may be chosen from cellulose ether derivatives comprising quaternary ammonium groups, cationic cellulose copolymers and cellulose derivatives grafted with a water-soluble quaternary ammonium monomer and cationic galactomannan gums.

The cellulose ether derivatives comprising quaternary ammonium groups are described in French Patent No. 1 492 597. These polymers are also defined in the CTFA dictionary as hydroxyethylcellulose quaternary ammoniums that have reacted with an epoxide substituted with a trimethylammonium group.

The cationic cellulose copolymers and cellulose derivatives grafted with a water-soluble quaternary ammonium monomer are described in U.S. Pat. No. 4,131,576, such as hydroxyalkylcelluloses, for instance hydroxymethyl-, hydroxyethyl- and hydroxypropylcelluloses grafted, for example, with a methacryloylethyltrimethylammonium, methacrylamidopropyltrimethylammonium and dimethyldiallylammonium salt.

The cationic galactomannan gums are described in U.S. Pat. Nos. 3,589,578 and 4 031 307, such as, for example, guar gums comprising trialkylammonium cationic groups. Use can be made, for example, of guar gums modified with a salt (e.g. chloride) of 2,3-epoxypropyltrimethylammonium.
(3) polymers comprising piperazinyl units and divalent alkylene and hydroxyalkylene radicals comprising straight and branched chains, optionally interrupted by oxygen, sulfur and nitrogen atoms and by aromatic and heterocyclic rings, and also the oxidation and/and quaternization products of these polymers. Such polymers are described, such as, for example, in French Patent Nos. 2 162 025 and 2 280 361;
(4) water-soluble polyamino amides prepared such as, for example, by polycondensation of an acidic compound with a polyamine; these polyamino amides may optionally be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide and optionally with an oligomer resulting from the reaction of a difunctional compound that is reactive with respect to a bis-halohydrin, a bis-azetidinium, a bis-haloacyldiamine, a bis-alkyl halide, an epihalohydrin, a diepoxide and a bis-unsaturated derivative; the crosslinking agent being used in an amount ranging from 0.025 to 0.35 mol per amine group of the polyamino amide; these polyamino amides may optionally be alkylated and, if they contain at least one tertiary amine functions, they may optionally be quaternized. Such polymers are described, such as, for example, in French Patent Nos. 2 252 840 and 2 368 508;
(5) polyamino amide derivatives resulting from the condensation of polyalkylene polyamines with polycarboxylic acids followed by alkylation with difunctional agents. Non-limiting mention may be made, for example, of adipic acid/dialkylamino-hydroxyalkyldialkylenetriamine polymers wherein the alkyl radical contains from 1 to 4 carbon atoms, for example, methyl, ethyl and propyl radicals. Such polymers are described, for example, in French Patent No. 1 583 363.

Among these derivatives, non-limiting mention may also be made of the adipic acid/dimethylaminohydroxypropyl/diethylenetriamine polymers sold under the name “Cartaretine F, F4 and F8” by the company Sandoz.
(6) polymers obtained by reaction of a polyalkylene polyamine comprising two primary amine groups and at least one secondary amine group with a dicarboxylic acid chosen from diglycolic acid and saturated aliphatic dicarboxylic acids ranging from 3 to 8 carbon atoms. The molar ratio from the polyalkylene polyamine and the dicarboxylic acid ranging from 0.8:1 to 1.4:1; the polyamino amide resulting therefrom being reacted with epichlorohydrin in a molar ratio of epichlorohydrin relative to the secondary amine group of the polyamino amide ranging from 0.5:1 to 1.8:1. Such polymers are described such as, for example, in U.S. Pat. Nos. 3,227,615 and 2 961 347.

Polymers of this type are sold such as, for example, under the name “Hercosett 57” by the company Hercules Inc. in the case of the adipic acid/epoxypropyl/diethylenetriamine copolymer.
(7) cyclopolymers of alkyldiallylamine and of dialkyldiallylammonium, such as the homopolymers and copolymers comprising, as main constituent of the chain, units chosen from those of formula (I) and/or (I′): 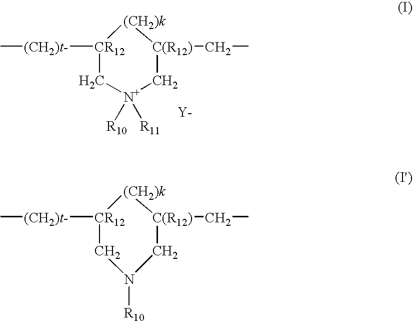
   wherein:
k and t are equal to 0 and 1, the sum k+t being equal to 1;
R₁₂ is chosen from a hydrogen atom and methyl radicals;
R₁₀ and R₁₁, which may be identical or different, are each chosen from alkyl groups comprising from 1 to 6 carbon atoms, hydroxyalkyl groups wherein the alkyl group may contain 1 to 5 carbon atoms, lower (C₁-C₄) amidoalkyl groups, and R₁₀ and R₁₁ may optionally each be chosen from, together with the nitrogen atom to which they are attached, heterocyclic groups such as piperidyl and morpholinyl; and
Y⁻ is an anion such as bromide, chloride, acetate, borate, citrate, tartrate, bisulfate, bisulfite, sulfate and phosphate.
   These polymers are described, for example, in French Patent No. 2 080 759 and in its Certificate of Addition No. 2 190 406.

In one embodiment of the present disclosure, R₁₀ and R₁₁, independently of each other, are each chosen from alkyl groups comprising from 1 to 4 carbon atoms.

Among the polymers defined above, non-limiting mention may also be made of the dimethyldiallylammonium chloride homopolymer sold under the name “Merquat 100” by the company Nalco (and its homologues of low weight-average molar mass) and copolymers of diallyidimethylammonium chloride and of acrylamide.
(8) diquaternary ammonium polymers comprising repeating units of formula (II):

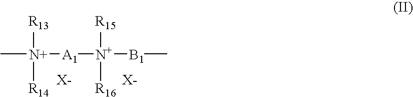

R₁₃, R₁₄, R₁₅ and R₁₆, which may be identical and different, are each chosen from aliphatic, alicyclic and arylaliphatic radicals comprising from 1 to 20 carbon atoms and lower hydroxyalkylaliphatic radicals, and R₁₃, R₁₄, R₁₅ and R₁₆, together and separately, constitute, with the nitrogen atoms to which they are attached, heterocycles optionally comprising a second hetero atom other than nitrogen, and alternatively R₁₃, R₁₄, R₁₅ and R₁₆ are chosen from a linear and branched C₁-C₆ alkyl radical substituted with a nitrile, ester, acyl and amide group and a group —CO—O—R₁₇-D and —CO—NH—R₁₇-D wherein R₁₇ is an alkylene and D is a quaternary ammonium group;
A₁ and B₁ are chosen from polymethylene groups comprising from 2 to 20 carbon atoms, which groups may be linear and branched, saturated and unsaturated, and which may contain, linked to and intercalated in the main chain, at least one aromatic rings and at least one oxygen and sulfur atoms and sulfoxide, sulfone, disulfide, amino, alkylamino, hydroxyl, quaternary ammonium, ureido, amide and ester groups, and
X⁻ is an anion derived from a mineral or organic acid;
A₁, R₁₃ and R₁ may optionally form, with the two nitrogen atoms to which they are attached, a piperazine ring; in addition, if A₁ is chosen from a linear and branched, saturated and unsaturated alkylene and hydroxyalkylene radicals, B₁ may optionally also be chosen from (CH₂)ₙ—CO-D-OC—(CH₂)ₙ—;
wherein:
D is chosen from:
a) a glycol residue of formula: —O-Z-O—, wherein Z is chosen from a linear and branched hydrocarbon-based radicals:
—(CH₂—CH₂-O)ₓ—CH₂-CH₂— radicals
—[CH₂—CH(CH₃)—O]_{y}—CH₂-CH(CH₃)— radicals
wherein x and y are each chosen from an integer from 1 to 4, representing a defined and unique degree of polymerization and any number from 1 to 4 representing an average degree of polymerization;
b) a bis-secondary diamine residue such as a piperazine derivative;
c) a bis-primary diamine residue of formula: —NH—Y—NH—, wherein Y is chosen from a linear and branched hydrocarbon-based radical, and the divalent radical —CH₂—CH₂-S—S—CH₂—CH₂—;
d) a ureylene group of formula: —NH—CO—NH—;
X⁻ is an anion such as, for example, chloride and bromide.

These polymers may have a number-average molar mass of ranging from 1000 to 100 000.

Polymers of this type are described such as, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is also possible to use polymers that comprise repeating units of formula (a): 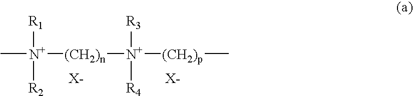
wherein R₁, R₂, R₃ and R₄, which may be identical and different, are each chosen from an alkyl and hydroxyalkyl radical comprising from 1 to 4 carbon atoms, n and p are integers ranging from 2 to 20, and X⁻ is an anion derived from a mineral and organic acid.

One compound of formula (a), for example, is one wherein R₁, R₂, R₃ and R₄ are methyl radicals and n=3, p=6 and X=Cl, which is known as Hexadimethrine chloride according to the INCI (CTFA) nomenclature.
(9) polyquaternary ammonium polymers comprise units of formula (III): 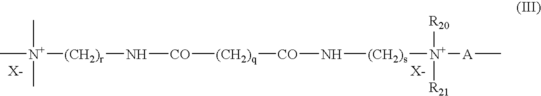
   R₁₈, R₁₉, R₂₀ and R₂₁, which may be identical and different, are each chosen from a hydrogen atom and methyl, ethyl, propyl, β-hydroxyethyl, β-hydroxypropyl and —CH₂CH₂(OCH₂CH₂)ₚOH radicals,
   wherein p is an integer ranging from 0 to 6, with the proviso that R₁₈, R₁₉, R₂₀ and R₂₁, are not simultaneously hydrogen atoms,
   r and s, which may be identical and different, are integers ranging from 1 to 6,
   q is an integer ranging from 0 to 34,
   X⁻ is an anion such as a halide,
   A is chosen from a dihalide radical and may be chosen from —CH₂—CH₂—O—CH₂—CH₂— radicals.

Such compounds are described, for example, in European Patent Application EP-A-122 324.

Among these products, non-limiting mention may be made, for example, of the products “Mirapol® A 15”, “Mirapol® AD1”, “Mirapol® AZ1” and “Mirapol® 175” sold by the company Miranol.
(10) quaternary polymers of vinylpyrrolidone and of vinylimidazole, for instance the products sold under the names Luviquat® FC 905, FC 550 and FC 370 by the company BASF.
(11) crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, and by copolymerization of acrylamide with dimethylaminoethyl methacrylate quaternized with methyl chloride, the homo- and copolymerization being followed by crosslinking with a compound comprising olefinic unsaturation, such as, for example, methylenebisacrylamide.

Other cationic polymers that may be used as disclosed herein are cationic proteins and cationic protein hydrolysates, polyalkyleneimines, such as, for example, polyethyleneimines, polymers comprising vinylpyridine and vinylpyridinium units, condensates of polyamines and of epichlorohydrin, polyquaternary ureylenes and chitin derivatives, such as, for example, chitosans and salts thereof;
the salts that may be used are, for example, chitosan acetate, lactate, glutamate, gluconate and pyrrolidonecarboxylate.

Among these compounds, non-limiting mention may be made of chitosan with a degree of deacetylation of 90% by weight, and the chitosan pyrrolidonecarboxylate sold under the name Kytamer® PC by the company Amerchol.

The cationic polymers that may be used in the context of the present disclosure may, for example, be chosen from the dimethyldiallylammonium chloride homopolymers and copolymers sold under the names “Merquat 100”, “Merquat 550” and “Merquat S” by the company Nalco, quaternary polymers of vinylpyrrolidone and of vinylimidazole, crosslinked homopolymers and copolymers of methacryloyloxy(C₁-C₄)alkyltri(C₁-C₄)alkylammonium salts, chitosan pyrrolidonecarboxylate and mixtures thereof.

The amphoteric polymers that may be used in accordance with the present disclosure may be chosen from polymers comprising units K and M randomly distributed in the polymer chain, wherein K is a unit derived from a monomer comprising at least one basic nitrogen atom and M is a unit derived from an acidic monomer comprising at least one carboxylic and sulfonic groups, and K and M may be chosen from groups derived from zwitterionic carboxybetaine and sulfobetaine monomers;
K and M may also be chosen from a cationic polymer chain comprising primary, secondary, tertiary and quaternary amine groups, wherein at least one of the amine groups bears a carboxylic and sulfonic group linked via a hydrocarbon-based radical, and
K and M form part of a chain of a polymer comprising an α,β-dicarboxylic ethylene unit wherein one of the carboxylic groups has been made to react with a polyamine comprising at least one amine group hosem from primary and secondary amine groups.

Among the amphoteric polymers that may be used as disclosed herein, non-limiting mention may be made of the following polymers:
(1) polymers resulting from the copolymerization of a monomer derived from a vinyl compound bearing a carboxylic group such as, acrylic acid, methacrylic acid, maleic acid, α-chloroacrylic acid, and a basic monomer derived from a substituted vinyl compound comprising at least one basic atom, such as dialkylaminoalkyl methacrylate and acrylate, dialkylaminoalkylmethacrylamide and -acrylamide. Such compounds are described in U.S. Pat. No. 3,836,537. Non-limiting mention may also be made of the sodium acrylate/acrylamidopropyltrimethylammonium chloride copolymer sold under the name Polyquart KE 3033 by the company Cognis.

The vinyl compound may also be a dialkyldiallylammonium salt such as dimethyldiallylammonium salt (for example chloride). The copolymers of acrylic acid and of the latter monomer are sold under the names Merquat 280 and Merquat 295 by the company Nalco.

(2) Polymers comprising units derived from:
a) at least one monomer chosen from acrylamides and methacrylamides substituted on the nitrogen with an alkyl radical,
b) at least one acidic comonomer comprising at least one reactive carboxylic groups, and
c) at least one basic comonomer such as esters comprising primary, secondary, tertiary and quaternary amine substituents of acrylic and methacrylic acids and the product of quaternization of dimethylaminoethyl methacrylate with dimethyl and diethyl sulfate.

The N-substituted acrylamides and methacrylamides may, for example, be chosen from groups wherein the alkyl radicals comprise from 2 to 12 carbon atoms, for example, N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen, for example, from acrylic acid, methacrylic acid, crotonic acid, itaconic acid, maleic acid and fumaric acid and alkyl monoesters, comprising 1 to 4 carbon atoms, of maleic and fumaric acids and anhydrides. Basic comonomers may be chosen, for example, from aminoethyl, butylaminoethyl, N,N′-dimethylaminoethyl and N-tert-butylaminoethyl methacrylates.

In one embodiment of the present disclosure, the copolymers whose CTFA (4th edition, 1991) name is Octylacrylamide/acrylates/butylaminoethyl methacrylate copolymer may, for example, be used.

(3) Polyamino amides that are crosslinked and alkylated partially and totally derived from polyamino amides of general formula (IV): [symbol]
CO—R₄—CO—Z [symbol] (IV)
wherein:
R₄ is chosen from a divalent radical derived from a saturated dicarboxylic acid, a mono- and dicarboxylic aliphatic acid comprising an ethylenic double bond, an ester of a lower alkanol, comprising from 1 to 6 carbon atoms of these acids and a radical derived from the addition of any one of said acids to a bis(primary) and bis(secondary) amine; and
Z is chosen from a bis(primary), mono- and bis(secondary) polyalkylene-polyamine radical and may be chosen from:
   a) in an amount ranging from 60 to 100 mol %, the radical
      —NH [symbol] (CH₂)ₓ—NH [symbol] ₚ (V)
   wherein x is equal to 2 and p is chosen from 2 and 3, or alternatively x is equal to 3 and p is equal to 2
   this radical being derived from diethylenetriamine, from triethylenetetraamine and from dipropylenetriamine;
   b) in an amount ranging from 0 to 40 mol %, the radical (V) above wherein x is equal to 2 and p is equal to 1 and which is derived from ethylenediamine, and radicals derived from piperazine: 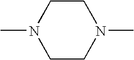
   c) in an amount ranging from 0 to 20 mol %, the —NH—(CH₂)₆—NH— radical derived from hexamethylenediamine, wherein the polyamino amines are crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid and an alkane sultone, and salts thereof.

The saturated carboxylic acids may, for example, be chosen from acids comprising from 6 to 10 carbon atoms, such as adipic acid, 2,2,4-trimethyladipic acid and 2,4,4-trimethyladipic acid, terephthalic acid and acids comprising an ethylenic double bond such as, for example, acrylic acid, methacrylic acid and itaconic acid.

The alkane sultones used in the alkylation can be chosen from, for example, propane sultone and butane sultone, and the salts of the alkylating agents may optionally be chosen from, for example, the sodium and potassium salts.

(4) Polymers comprising zwitterionic units of formula (VI): 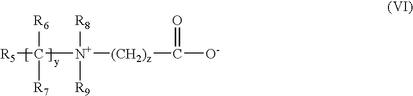
wherein:
R₅ is chosen from a polymerizable unsaturated group chosen from, for example, acrylate, methacrylate, acrylamide and methacrylamide groups;
y and z are integers ranging from 1 to 3;
R₆ and R₇ are chosen from a hydrogen atom, methyl, ethyl and propyl groups;
R₈ and R₉ are chosen from a hydrogen atom and alkyl radicals such that the sum of the carbon atoms in R₈ and R₉ does not exceed 10.

The polymers comprising such units may also comprise units derived from non-zwitterionic monomers such as dimethyl and diethylaminoethyl acrylate and methacrylate, and alkyl acrylates and methacrylates, acrylamides and methacrylamides, and vinyl acetate.

By way of non-limiting example, mention may be made of the copolymer of butyl methacrylate/dimethylcarboxymethylammonioethyl methacrylate.

(5) Polymers derived from chitosan comprising monomer units chosen from those of formulae (VII), (VIII) and (IX): 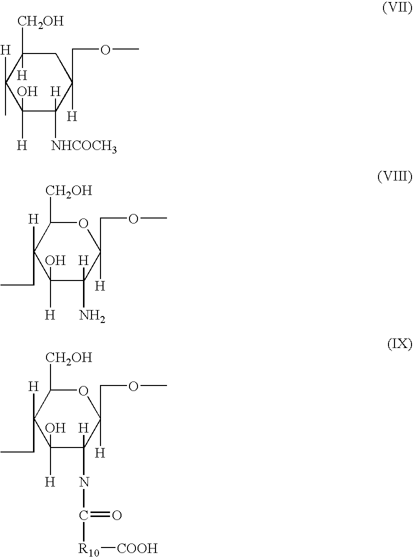
wherein:
the unit (VII) is present in an amount ranging from 0 and 30%;
the unit (VIII) is present in an amount ranging from 5 to 50%; and
the unit (IX) is present in an amount ranging from 30 to 90%, it being understood that, in this unit (IX), R₁₀ is chosen from a radical of formula: 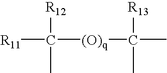
   wherein”
if q=0, R₁₁, R₁₂ and R₁₃, which may be identical and different, each are chosen from a hydrogen atom, a methyl, hydroxyl, acetoxy and amino residues, monoalkylamine residues and dialkylamine residues that are optionally interrupted by at least one nitrogen atom and/and optionally substituted with at least one entity chosen from amine, hydroxyl, carboxyl, alkylthio and sulfonic groups, and alkylthio radicals wherein the alkyl group bears an amino residue, and wherein at least one of the radicals R₁₁, R₁₂ and R₁₃ is a hydrogen atom;
and, if q is equal to 1, R₁₁, R₁₂ and R₁₃ are each a hydrogen atom, and also the salts formed by these compounds with bases and acids.

(6) Polymers derived from the N-carboxyalkylation of chitosan, such as N-carboxymethylchitosan and N-carboxybutylchitosan.

(7) Polymers of formula (X) such as those described, for example, in French Patent No. 1 400 366: 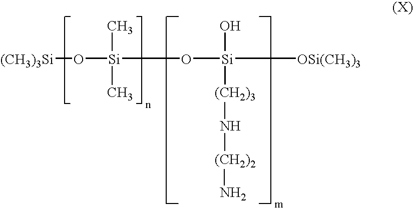
r is an integer greater than 1, R₁₄ is chosen from a hydrogen atom, a CH₃O, CH₃CH₂O and phenyl radical, R₁₅ is chosen from hydrogen atoms and lower alkyl radicals such as methyl and ethyl, R₁₆ is chosen from hydrogen atoms and lower alkyl radicals such as methyl and ethyl, R₁₇ is chosen from lower alkyl radicals such as methyl and ethyl and —R₁₈—N(R₁₆)₂ radicals, wherein:
R₁₈ chosen from —CH₂—CH₂—, —CH₂—CH₂-CH₂— and —CH₂—CH(CH₃)— groups,
R₁₆ has the meanings mentioned above, and also the higher homologues of these radicals and comprising up to 6 carbon atoms.

(8) Amphoteric polymers of the type -D-X-D-X- chosen from:
a) polymers obtained by the action of chloroacetic acid and sodium chloroacetate on compounds comprising at least one unit of formula (XI):
   -D-X-D-X-D- (XI)
   wherein D is the radical: 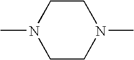
and X is chosen from E and E′, wherein E and E′, which may be identical and different, are divalent radicals chosen from straight or branched alkylene radicals comprising up to 7 carbon atoms in the main chain, which can be unsubstituted and substituted with hydroxyl groups and that may also comprise at least one heteroatom chosen from oxygen, nitrogen and sulfur atoms, and may also comprise 1 to 3 aromatic and/or heterocyclic rings; wherein the oxygen, nitrogen and sulfur atoms are present in a form chosen from ether, thioether, sulfoxide, sulfone, sulfonium, alkylamine, alkenylamine groups, hydroxyl, benzylamine, amine oxide, quaternary ammonium, amide, imide, alcohol, ester and urethane groups;
   b) polymers of formula (XII):
      -D-X-D-X- (XII)
      wherein D is the radical 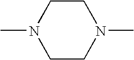
and X is chosen from E and E′ and at least once E′; wherein E has the meaning given above and E′ is a divalent radical chosen from straight and branched alkylene radicals comprising up to 7 carbon atoms in the main chain, which are unsubstituted or substituted with at least one hydroxyl radical and comprising at least one nitrogen atom, wherein the nitrogen atom is substituted with an alkyl chain that is optionally interrupted by an oxygen atom and comprises at least one functional group chosen from carboxyl and hydroxyl functional groups, and betainized by reaction with chloroacetic acid and sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine and by semiesterification with an N,N-dialkanolamine. These copolymers may optionally also comprise other vinyl comonomers such as vinylcaprolactam.

In one embodiment of the present disclosure, the amphoteric polymers of family (1) are used.

According to the present disclosure, the at least one additional cationic or amphoteric polymer can be present in a total amount ranging from 0.001% to 20% by weight, for example, from 0.01% to 10% by weight and further, for example, from 0.02% to 5% by weight, relative to the total weight of the final composition.

According to one embodiment of the present disclosure, the compositions may also comprise at least one silicone other than the aminosilicones of the present disclosure.

Among the silicones that may be used in the compositions of the present disclosure, non-limiting mention may be made, for example, of volatile and non-volatile, cyclic and acyclic, branched and unbranched, organomodified and non-organomodified silicones, as described below.

The silicones that may be used as disclosed herein may be soluble and insoluble in the composition, and for example, may be chosen from polyorganosiloxanes that are insoluble in the composition of the present disclosure; they may be in the form of oils, waxes, resins and gums.

According to the present disclosure, all the silicones may be used in unmodified form and in the form of solutions, dispersions, emulsions, nanoemulsions and microemulsions.

The organopolysiloxanes are defined, for example, in Walter Noll's “Chemistry and Technology of Silicones” (1968) Academic Press. They may be volatile and non-volatile.

When they are volatile, the silicones may be chosen, for example, from those having a boiling point of from 60° C. and 260° C. such as from:
(i) cyclic silicones comprising from 3 to 7, for example, 4 to 5 silicon atoms. These are, for example, octamethylcyclotetrasiloxane sold, for example, under the name “Volatile Silicone 7207” by Union Carbide and “Silbione 70045 V 2” by Rhodia, decamethylcyclopentasiloxane sold under the name “Volatile Silicone 7158” by Union Carbide, and “Silbione 70045 V 5” by Rhodia, and mixtures thereof.

Non-limiting mention may also be made of cyclocopolymers of the dimethylsiloxane/methylalkylsiloxane type, such as “Volatile Silicone FZ 3109” sold by the company Union Carbide, having the chemical structure:

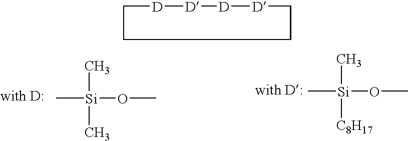

Non-limiting mention may further be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;
(ii) linear volatile silicones comprising 2 to 9 silicon atoms and having a viscosity of less than and equal to 5×10⁻⁶ m²/s at 25° C. An example is decamethyl-tetrasiloxane sold, for example, under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

In one aspect of the present disclosure, non-volatile silicones may be used, for example, polyalkylsiloxanes, polyarylsiloxanes, polyalkylarylsiloxanes, silicone gums, silicone resins, and polyorganosiloxanes modified with organofunctional groups, and also mixtures thereof. These silicones may be chosen from polyalkylsiloxanes, among which non-limiting mention may be made of polydimethylsiloxanes comprising trimethylsilyl end groups and having a viscosity of from 5×10⁻⁶ to 2.5 m²/s at 25° C., for example, 1×10⁻⁵ to 1 m²/s. The viscosity of the silicones is measured, for example, at 25° C. according to ASTM standard 445 Appendix C.

Among these polyalkylsiloxanes, mention may be made, in a non-limiting manner, of the following commercial products:
the Silbione® oils of the 47 and 70 047 series and the Mirasil® oils sold by Rhodia, such as, for example, the oil 70 047 V 500 000;
the oils of the Mirasil® series sold by the company Rhodia;
the oils of the 200 series from the company Dow Corning, such as, DC200 with a viscosity of 60 000 mm²/s;
the Viscasil® oils from General Electric and certain oils of the SF series (SF 96, SF 18) from General Electric.

Non-limiting mention may also be made of polydimethylsiloxanes comprising dimethylsilanol end groups, known under the name dimethiconol (CTFA) such as the oils of the 48 series from the company Rhodia.

In this category of polyalkylsiloxanes, non-limiting mention may also be made of the products sold under the names “Abil Wax® 9800 and 9801” by the company Goldschmidt, which are poly(C₁-C₂₀)alkylsiloxanes.

The polyalkylarylsiloxanes may, for example, be chosen from polydimethyl/methylphenylsiloxanes, and linear and branched polydimethyldiphenylsiloxanes with a viscosity ranging from 1×10⁻⁵ to 5×10⁻² m²/s at 25° C.

Among these polyalkylarylsiloxanes, non-limiting examples that may be mentioned comprise the products sold under the following names:
the Silbione® oils of the series 70 641 from Rhodia;
the oils of the Rhodorsil® 70 633 and 763 series from Rhodia;
the oil Dow Corning 556 Cosmetic Grade Fluid from Dow Corning;
the silicones of the PK series from Bayer, for instance the product PK20;
the silicones of the PN and PH series from Bayer, for instance the products PN1000 and PH1000;
certain oils of the SF series from General Electric, such as SF 1023, SF 1154, SF 1250 and SF 1265.

The silicone gums that may be used in accordance with the present disclosure are, for example, polydiorganosiloxanes having high number-average molecular masses ranging from 200 000 and 1 000 000, used alone and as a mixture in a solvent. This solvent may be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, and mixtures thereof.

Non-limiting mention may be made of the following products:
polydimethylsiloxane,
polydimethylsiloxane/methylvinylsiloxane gums,
polydimethylsiloxane/diphenylsiloxane,
polydimethylsiloxane/phenylmethylsiloxane,
polydimethylsiloxane/diphenylsiloxane/methylvinylsiloxane.

Further non-limiting examples of products that may be used in accordance with the present disclosure are mixtures such as:
mixtures formed from a polydimethylsiloxane hydroxylated at the chain end, and dimethiconol (CTFA), and from a cyclic polydimethylsiloxane, also known as cyclomethicone (CTFA), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs of different viscosities, for example, a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product may contain 15% SE 30 gum and 85% SF 96 oil.

The organopolysiloxane resins that may optionally be used in accordance with the present disclosure are crosslinked siloxane systems comprising the following units:

R₂SiO_{2/2}, R₃SiO_{1/2}, RSiO_{3/2} and SiO_{4/2} wherein R is chosen from hydrocarbon-based groups comprising 1 to 16 carbon atoms and phenyl groups, for example wherein R is chosen from C₁-C₄ lower alkyl radicals, and further, for example, methyl and phenyl radicals.

Among these resins, non-limiting mention may be made of the product sold under the name “Dow Corning 593” and those sold under the names “Silicone Fluid SS 4230 and SS 4267” by the company General Electric, which are silicones of dimethyl/trimethyl siloxane structure.

Non-limiting mention may also be made of the trimethyl siloxysilicate type resins sold, for example, under the names X22-4914, X21-5034 and X21-5037 by the company Shin-Etsu.

The organomodified silicones that may, for example, be used in accordance with the present disclosure are silicones as defined above and comprising in their structure at least one organofunctional groups attached via a hydrocarbon-based group.

Among the organomodified silicones, non-limiting mention may be made of polyorganosiloxanes comprising:
polyethyleneoxy and/and polypropyleneoxy groups optionally comprising C₆-C₂₄ alkyl groups, such as the products known as dimethicone copolyol sold by the company Dow Corning under the name DC 1248 and the oils Silwet® L 722, L 7500, L 77 and L 711 by the company Union Carbide, and the (C₁₂)alkylmethicone copolyol sold by the company Dow Corning under the name Q2 5200;
thiol groups such as the products sold under the names “GP 72 A” and “GP 71” from Genesee;
alkoxylated groups such as the product sold under the name “Silicone Copolymer F-755” by SWS Silicones and Abil Wax® 2428, 2434 and 2440 by the company Goldschmidt;
hydroxylated groups such as the polyorganosiloxanes containing a hydroxyalkyl function, described in French Patent Application FR-A-85/16334;
acyloxyalkyl groups such as, for example, the polyorganosiloxanes described in U.S. Pat. No. 4,957,732 A;
anionic groups of carboxylic type, such as, for example, in the products described in European Patent No. EP 186 507 from the company Chisso Corporation, and of alkylcarboxylic type, such as those present in the product X-22-3701 E from the company Shin-Etsu; 2-hydroxyalkyl sulfonate; 2-hydroxyalkyl thiosulfate such as the products sold by the company Goldschmidt under the names “Abil® S201” and “Abil® S255”;
hydroxyacylamino groups, such as the polyorganosiloxanes described in European Patent Application EP 342 834. Mention may be made, for example, of the product Q2-8413 from the company Dow Corning.

The silicones as described above may be used alone and as a mixture, in an amount ranging from 0.01% to 20% by weight, for example, ranging from 0.05% to 5% by weight relative to the total weight of the composition.

As used herein the term “cosmetically acceptable medium” is understood to mean a medium that is compatible with keratin materials such as, for example, the skin, the eyelashes and the hair.

The cosmetically acceptable medium may be solely water or comprise a mixture of water and of a cosmetically acceptable solvent such as a C₁-C₄ lower alcohol, for instance ethanol, isopropanol, tert-butanol and n-butanol; alkylene glycols, for instance propylene glycol, and polyol ethers; and mixtures thereof.

The composition may, for example, comprise from 50% to 95% by weight of water, relative to the total weight of the composition.

The washing compositions according to the disclosure have a final pH ranging from 3 to 10, for example, from 4.5 to 8. The pH may be adjusted to the desired value conventionally, by adding a base (organic and mineral base) to the composition, for example aqueous ammonia and a primary, secondary and tertiary (poly)amine, for instance monoethanolamine, diethanolamine, triethanolamine, isopropanolamine and 1,3propanediamine, and by adding an acid such as, for example, a carboxylic acid, and further, for example, citric acid.

The compositions as disclosed herein may further comprise viscosity regulators such as electrolytes, and thickeners (associative and non-associative thickeners). Non-limiting mention may be made, for example, of sodium chloride, sodium xylenesulfonate, scleroglucans, xanthan gums, fatty acid alkanolamides, alkyl ether carboxylic acid alkanolamides optionally oxyethylenated with up to 5 mol of ethylene oxide, such as the product sold under the name “Aminol A15” by the company Chem Y, crosslinked polyacrylic acids and acrylic acid copolymers such as crosslinked acrylic acid/C₁₀-C₃₀ alkyl acrylate copolymers. These viscosity regulators may be used in the compositions according to the disclosure in a total amount ranging from 0 to 10% by weight, relative to the total weight of the composition.

The compositions in accordance with the disclosure may also, for example, comprise up to 5% of nacreous agents and opacifiers such as, for example, C16 higher fatty alcohols, fatty-chain acyl derivatives such as ethylene glycol and polyethylene glycol monostearate and distearate, and fatty-chain (C₁₀-C₃₀) ethers such as, for example, distearyl ether and 1-(hexadecyloxy)-2-octadecanol.

The compositions in accordance with the disclosure may, for example, also contain at least one additive chosen from foam synergists such as C₁₀-C₁₈ 1,2-alkanediols and fatty alkanolamides derived from monoethanolamine and diethanolamine; silicone and non-silicone sunscreens; anionic and nonionic polymers; cationic surfactants; proteins; protein hydrolysates; ceramides; pseudoceramides; linear and branched C₁₂-C₄₀ fatty acids such as 18-methyleicosanoic acid; hydroxy acids; vitamins; provitamins such as panthenol; animal, mineral and synthetic oils; and any other additive conventionally used in cosmetics that does not affect the properties of the compositions disclosed herein.

The washing compositions according to the disclosure may also comprise at least one adjuvant usually encountered in the field of shampoos, for instance fragrances, preserving agents, sequestering agents, softeners, dyes, moisturizers, antidandruff agents and anti-seborrhoeic agents, and the like.

A person skilled in the art will take care to select this and these optional additional compound(s) and/and the amounts thereof such that the beneficial properties intrinsically associated with the combination in accordance with the present disclosure are not, and are not substantially, adversely affected by the envisioned addition(s).

These compositions may be in the form of optionally thickened liquids, creams and gels, for example, in a form suitable for washing, caring for and/and styling the hair.

The present disclosure also relates to a cosmetic process for treating keratin materials, which comprises applying an effective amount of a composition as described above to the keratin materials and rinsing after an optional leave-in time.

According to one embodiment of the present disclosure, the composition may be used as a shampoo.

When the compositions in accordance with the disclosure are used as standard shampoos, they can simply be applied to wet hair and the lather generated by massaging and friction with the hands is then removed, after an optional action time, by rinsing with water, the operation possibly being repeated one or more times.

Other than in the operating examples, and where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The following examples are intended to illustrate the invention in a non-limiting manner.

### EXAMPLES 1 to 3

Shampoo compositions in accordance with the disclosure were prepared:

| Constituent | 1 | 2 | 3 |
|---|---|---|---|
| Copolymer (90 mol/10 mol) of | 0.6% AM | 0.5% AM | — |
| acrylamide and of dimethyl- | | | |
| aminoethyl acrylate quaternized | | | |
| with benzyl chloride | | | |
| (MW > 5 × 10⁶) as a | | | |
| dispersion in a concentrated saline | | | |
| aqueous solution (Ultimer from | | | |
| Ondeo) | | | |
| Copolymer (10 mol/90 mol) of | — | — | 0.5% AM |
| dimethylaminoethyl acrylate | | | |
| quaternized with methyl chloride | | | |
| and of acrylamide (MW >5 × 10⁶) | | | |
| as a dispersion in a concentrated | | | |
| saline aqueous solution | | | |
| Sodium lauryl ether sulfate 2.2 EO | 15% AM | 13% AM | 12.5% AM |
| Cocoamidopropylbetaine | 5% AM | 4% AM | 2.5% AM |

| | | | |
|---|---|---|---|
| Amodimethicone | 1% | — | 0.8% |
| (DC 939 from Dow Corning - 35% AM) | AM | | AM |
| Chitosan PCA | — | 0.3% | 0.2% |
| (Kytamer PC from Amerchol) | | | |
| Trimethylsilyl amodimethicone | — | 1% | — |
| (DC2 - 8566 Amino Fluid from | | | |
| Dow Corning) | | | |
| Ethanol | — | 1% | — |
| NaCl | — | — | 2% |
| Citric acid and NaOH qs | pH 6.5 | pH 6.5 | pH 6.5 |
| Water qs | 100% | 100% | 100% |

Hair treated with these shampoos had good styling and volumizing properties.

1. A composition for washing keratin materials comprising, in a cosmetically acceptable aqueous medium,
at least one aminosilicone,
at least one detergent surfactant chosen from anionic, nonionic and amphoteric surfactants and
at least one drawing polymer with a drawing power of greater than 5 cm.

2. A composition according to claim 1, wherein the at least one detergent surfactant is chosen from anionic surfactants.

3. A composition according to claim 1, wherein the at least one detergent surfactant is present in an amount ranging from 4% to 50% relative to the total weight of the composition.

4. A composition according to claim 3, wherein the at least one detergent surfactant is present in an amount ranging from 8% to 25% relative to the total weight of the composition.

5. A composition according to claim 1, wherein the at least one drawing polymer with a drawing power of greater than 5 cm is chosen from either:
(a1) a dispersion of particles of at least one water-soluble polymer with a weight-average molecular mass of greater than 10⁶ in a saline aqueous solution, obtained by heterogeneous free-radical polymerization of water-soluble monomers with precipitation of the polymer formed, at least one of the monomers being cationic, or
(a2) a saline aqueous solution of at least one water-soluble polymer with a weight-average molecular mass of greater than 10⁶, obtained by heterogeneous free-radical polymerization of water-soluble monomers with precipitation of the polymer formed, at least one of the monomers being cationic.

6. A composition according to claim 5, wherein the at least one water-soluble monomer is chosen from cationic, anionic and nonionic monomers comprising at least one ethylenic double bond.

7. A composition according to claim 6, wherein the anionic monomers are chosen from acrylic acid, methacrylic acid, acrylamido-2-methylpropanesulfonic acid and itaconic acid.

8. A composition according to claim 6, wherein the nonionic monomers are chosen from acrylamide, methacrylamide, N-vinylformamide, N-vinylacetonamide, hydroxypropyl acrylate and hydroxypropyl methacrylate.

9. A composition according to claim 6, wherein the cationic monomers are chosen from di(C₁₋₄ alkyl)diallylammonium salts and the compounds of formula (I) 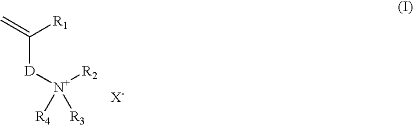
wherein:
R₁ is chosen from a hydrogen atom and methyl groups;
R₂ and R₃, which may be identical or different, are chosen from a hydrogen atom and linear and branched C₁₋₄ alkyl groups;
R₄ is chosen from a hydrogen atom, linear and branched C₁₋₄ alkyl groups and aryl groups;
D is chosen from the following unit 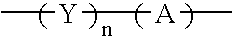
wherein:
Y is chosen from amide, ester, urethane and urea functional groups;
A is chosen from linear, branched and cyclic C₁₋₁₀ alkylene groups, optionally substituted and interrupted with a ring chosen from divalent aromatic and heteroaromatic rings, and optionally interrupted with a heteroatom chosen from O, N, S and P, and which optionally may comprise a functional group chosen from ketone, amide, ester, urethane and urea functional groups;
n is equal to 0 or 1; and
X⁻ is an anionic counterion.

10. A composition according to claim 5, wherein the at least one water-soluble polymer is obtained by heterogeneous free-radical polymerization of at least one cationic monomer of formula (I).

11. A composition according to claim 10, wherein the at least one water-soluble polymer is obtained by heterogeneous free-radical polymerization of a monomer mixture comprising
from 0 to 30 mol % of acrylic acid,
from 0 to 95.5 mol % of acrylamide and
from 0.5 to 100 mol % of at least one cationic monomer of formula (I).

12. A composition according to claim 11, wherein the at least one water-soluble polymer is obtained by heterogeneous free-radical polymerization of a monomer mixture comprising at least one acrylic acid and at least one cationic monomer of formula (I), the number of moles of the cationic monomer of formula (I) being greater than the number of moles of acrylic acid.

13. A composition according to claim 11, wherein the at least one water-soluble polymer is obtained by heterogeneous free-radical polymerization of a monomer mixture comprising
10 mol % of acryloyloxyethyldimethylbenzylammonium chloride and
90 mol % of acrylamide.

14. A composition according to claim 11, wherein the at least one water-soluble polymer is obtained by heterogeneous free-radical polymerization of a monomer mixture comprising
30 mol % of acryloyloxytrimethylammonium chloride,
50 mol % of acryloyloxyethyldimethylbenzylammonium chloride and
20 mol % of acrylamide.

15. A composition according to claim 11, wherein the at least one water-soluble polymer is obtained by heterogeneous free-radical polymerization of a monomer mixture comprising
10 mol % of acryloyloxyethyltrimethylammonium chloride and
90 mol % of acrylamide.

16. A composition according to claim 11, wherein the at least one water-soluble polymer is obtained by heterogeneous free-radical polymerization of a monomer mixture comprising
30 mol % of diallyldimethylammonium chloride and
70 mol % of acrylamide.

17. A composition according to claim 5, wherein the concentration of particles of at least one water-soluble polymer present as a dispersion in a saline aqueous solution in (a1) ranges from 0.01% to 20% by weight, relative to the total weight of the dispersion.

18. A composition according to claim 5, wherein the concentration of the at least one water-soluble polymer present as a solution in (a2) ranges from 0.01% to 20% by weight, relative to the total weight of the solution.

19. A composition according to claim 5, wherein the saline aqueous solution in (a1) and (a2) contains at least one anionic salt.

20. A composition according to claim 19, wherein the at least one anionic salt is chosen from ammonium sulfate, ammonium hydrogen sulfate, sodium sulfate, sodium hydrogen sulfate, magnesium sulfate, ammonium sulfate, magnesium hydrogen sulfate, aluminium sulfate and aluminium hydrogen sulfate.

21. A composition according to claim 1, wherein the at least one drawing polymer is present in an amount ranging from 0.01% to 10% by weight relative to the total weight of the composition.

22. A composition according to claim 1, wherein the at least one aminosilicone is chosen from:
(a) the amodimethicone polysiloxanes of formula (VIII): 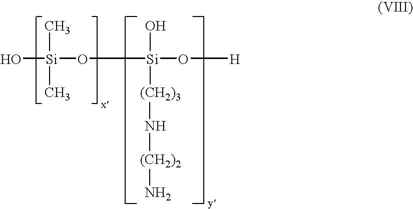
wherein x′ and y′ are integers such that the number-average molecular weight ranges from 5000 to 500 000;
(b) aminosilicones of formula (IX):
RₐG₃₋ₐ-Si(OSiG₂)ₙ—(OSiG_{b}R′_{2-b})ₘ—O—SiG₃₋ₐ-R′ₐ (IX)
wherein:
G is chosen from a hydrogen atom, OH groups, phenyl groups, and C₁-C₈ alkyl groups;
a is an integer ranging from 0 to 3;
b is equal to 0 or 1;
m and n are numbers such that the sum (n+m) ranges from 1 to 2000, n is an integer ranging from 0 to 1999, and m is an integer ranging from 1 to 2000;
R′ is a monovalent radical of formula
—C_{q}H_{2q}L
wherein:
q is an integer ranging from 2 to 8; and
L is an optionally quaternized amine group chosen from:
—NR″-CH₂—CH₂-N′(R″)₂
—N(R″)₂
—N⊕(R″)₃A⁻
—NH⊕(R″)₂A⁻
—NH₂⊕(R″)A⁻
—N(R″)—CH₂—CH₂-N⊕R″H₂A^{-,}
wherein:
R″ is chosen from hydrogen, phenyl, benzyl and a saturated monovalent hydrocarbon-based radical; and
A⁻ is a halide ion;
(c) aminosilicones of the formula (XI): 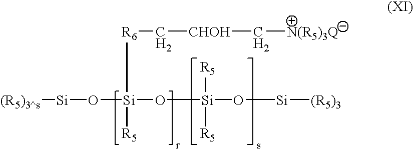
wherein:
R₅ is chosen from monovalent hydrocarbon-based radicals comprising from 1 to 18 carbon atoms;
R₆ is chosen from divalent hydrocarbon-based radicals;
Q⁻ is chosen from anions and organic acid salts;
r is a number ranging from 2 to 20; and
s is a number ranging from 20 to 200;
d) quaternary ammonium silicones of formula (XII): 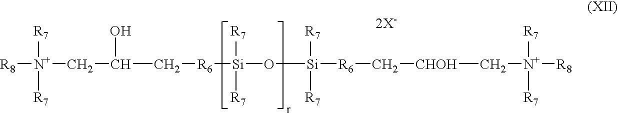
wherein:
R₇, which may be identical and different, is chosen from monovalent hydrocarbon-based radicals comprising from 1 to 18 carbon atoms.
R₆ is a divalent hydrocarbon-based radical;
R₈, which may be identical and different, is chosen from hydrogen atoms, monovalent hydrocarbon-based radicals comprising from 1 to 18 carbon atoms, C₂-C₁₈ alkenyl radicals and radical —R₆—NHCOR₇;
X⁻ is chosen from an anion and an organic acid salt;
r is a number ranging from 2 to 200;
e) aminosilicones of formula (XIII): 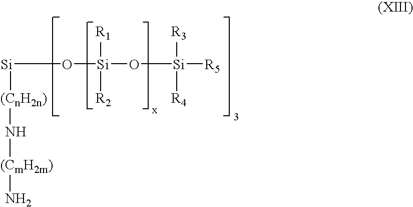
R₁, R₂, R₃ and R₄, which may be identical and different, are each chosen from C₁-C₄ alkyl radicals and phenyl groups,
R₅ is chosen from C₁-C₄ alkyl radicals and hydroxyl groups,
n is an integer ranging from 1 to 5,
m is an integer ranging from 1 to 5,
and wherein x is chosen such that the amine number ranges from 0.01 to 1 meq/g.

23. A composition according to claim 1, wherein the at least one aminosilicone is present in an amount ranging from 0.001% to 20% by weight relative to the total weight of the composition.

24. A composition according to claim 1, wherein the composition also comprises at least one additional cationic polymer.

25. A composition according to claim 24, wherein the at least one additional cationic polymer is chosen from cationic cyclopolymers, quaternary polymers of vinylpyrrolidone and of vinylimidazole, crosslinked homopolymers and copolymers of methacryloyloxy(C₁-C₄)alkyltri(C₁-C₄)alkylammonium salts, and chitosan pyrrolidonecarboxylate.

26. A composition according to claim 24, wherein the at least one additional cationic polymer is present in an amount ranging from 0.001% to 20% by weight relative to the total weight of the final composition.

27. A composition according to claim 1, wherein the composition also comprises at least one silicone other than aminosilicones.

28. A composition according to claim 27, wherein the at least one silicone other than aminosilicone is present in an amount ranging from 0.01% to 20% by weight relative to the total weight of the composition.

29. A composition according to claim 1, wherein the composition has a pH ranging from 3 to 10.

30. A process for cleansing and/and caring for and/and conditioning and/and styling keratin fibers, comprising applying to said keratin fibers a composition comprising, in a cosmetically acceptable aqueous medium,
at least one aminosilicone,
at least one detergent surfactant chosen from anionic, nonionic and amphoteric surfactants and
at least one drawing polymer with a drawing power of greater than 5 cm.
